(19) United States
(12) Patent Application Publication
(10) Pub. No.: US 2010/0094277 A1
(43) Pub. Date: Apr. 15, 2010

(54) HIGH-FREQUENCY SURGICAL DEVICE AND METHOD

(75) Inventors: Taisuke SATO, Tokyo (JP); Manabu ISHIKAWA, Tokyo (JP); Makoto INABA, Tokyo (JP); Takashi MIHORI, Tokyo (JP); Yoshitaka HONDA, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/248,523

(22) Filed: Oct. 9, 2008

(51) Int. Cl. A61B 18/14

(52) U.S. Cl. 606/41

(57) ABSTRACT

A high-frequency surgical device includes: a treatment section provided with electrodes for supplying high-frequency power to living tissue of a patent foramen ovale; a high-frequency power supplying section for supplying high-frequency power to living tissue around the electrodes through the electrodes; a blood flow detecting section for detecting intracardiac blood flow information based on biological information inputted from outside; and a control section for controlling high-frequency power to be supplied to the side of electrodes based on the blood flow information.

### BACKGROUND OF THE INVENTION

1. Field of the Invention

The present invention relates to a high-frequency surgical device and method for performing high-frequency surgery by supplying high-frequency current to living tissue to be treated.

2. Description of the Related Art

High-frequency surgical devices are generally known to perform high-frequency surgery by utilizing high-frequency power, to fuse a portion of living tissue with another portion of living tissue. The principle of such a high-frequency surgical device is provided below.

When high-frequency power is applied to living tissue, the tissue is warmed by the Joule heat of the tissue per se. Living tissue, when sufficiently warmed, is inherently degenerated and fuses with another portion of living tissue. When both portions of the tissue are pressed and brought into contact with each other in the sufficiently warmed state, both portions of the tissue will fuse with each other. Accordingly, when fusion is desired to be attained between portions of tissue, the portions of tissue should be pressed and contacted with each other with the application of high-frequency power. The Joule heat is known to become higher as the density of high-frequency current becomes higher.

Some well-known high-frequency devices utilize the above principle to occlude a patent foramen ovale (PFO). The PFO is a flap-shaped gap present in a portion of atrial septa which space apart a right atrium of a heart from a left atrium. Generally, the left atrium has a higher pressure than the right atrium, and thus a valve of oval foramen is pressurized and in contact with the atrial septa to close the PFO. However, when a person has a severe cough or is nervous (when the person's lungs are pressurized), for example, the pressure difference may be reversed to temporarily open the flap. At this instant, blood clots that have flowed into the right atrium may likely to pass through the FPO to directly reach the brain, inducing cerebral infarction. For this reason, desirably, the PFO should be occluded.

For example, PCT Publication No. WO2004/086944 discloses a high-frequency device for treating a PFO. In the prior art technique disclosed in the literature, the treatment for occluding a PFO is given by sandwiching the PFO, from its lateral sides, between two high-frequency electrodes, followed by applying high-frequency power to the PFO.

In applying high-frequency power to a PFO for occlusion treatment of the PFO, it is desired that the occlusion treatment is given with the control of the high-frequency power so that no blood clot is caused.

### SUMMARY OF THE INVENTION

A high-frequency surgical device of the present invention includes:

a treatment section provided with electrodes for supplying high-frequency power to living tissue of a patent foramen ovale;

a high-frequency power supplying section for supplying high-frequency power to living tissue around the electrodes through the electrodes;

a biological information inputting section for inputting biological information from outside;

a blood flow detecting section for detecting intracardiac blood flow information, based on the biological information inputted from the biological information inputting section; and

a control section for controlling high-frequency power to be supplied to the electrodes, based on the intracardiac blood flow information detected by the blood flow detecting section.

A high-frequency surgical method related to the present invention for giving a treatment of occlusion to a patent foramen ovale by supplying high-frequency power to living tissue of the patent foramen ovale through electrodes provided at a distal end side of a high-frequency probe, includes:

a step of positioning and setting electrodes provided at a distal end side of a high-frequency probe, in living tissue of a patent foramen ovale to be treated;

a step of inputting biological information;

a step of detecting timings of the biological information corresponding to a period when blood flow velocity or blood flow volume is large, based on results of detection for the inputted biological information; and

a step of effecting control for supplying the high-frequency power to the electrodes during the period when blood flow velocity or blood flow volume is large, according to the step of detecting timing.

### BRIEF DESCRIPTION OF THE DRAWINGS

FIG. 1 is a schematic diagram illustrating a general configuration of a high-frequency surgical device according to a first embodiment of the present invention;

FIG. 2 illustrates a partially cut out structure on a distal end side of a high-frequency probe;

FIG. 3 is a schematic diagram illustrating an electrical system of the high-frequency surgical device including a high-frequency power unit;

FIG. 4 is a block diagram illustrating a peak detection circuit configuring a blood flow detecting section;

FIG. 5 is an explanatory view illustrating a vicinity of a PFO in a heart;

FIG. 6A is a diagram illustrating the vicinity of the PFO with the high-frequency probe being positioned at the PFO;

FIG. 6B is a diagram illustrating a vicinity of a distal end portion of the high-frequency probe, as viewed from a direction “A” in FIG. 6A;

FIG. 7 is a timing diagram illustrating a relationship of an electrocardiographic waveform with respect to start and stoppage of supplying high-frequency power;

FIG. 8 is a flow diagram illustrating an example of a procedure of a high-frequency surgical method according to the first embodiment;

FIG. 9 is a schematic diagram illustrating an electrical system of a high-frequency surgical device according to a second embodiment of the present invention;

FIG. 10 is a schematic diagram illustrating a case where an intracardiac color Doppler image is displayed, using an ultrasound probe;

FIG. 11 is a flow diagram illustrating an example of a procedure for a high-frequency surgical method according to the second embodiment;

FIG. 12 is a diagram illustrating a distal end side of a high-frequency probe according to a third embodiment of the present invention;

FIG. 13 is an enlarged cross-sectional view illustrating the distal end side of the high-frequency probe illustrated in FIG. 12;

FIG. 14 is a schematic diagram illustrating an electrical system of a high-frequency surgical device according to the third embodiment;

FIG. 15 is a timing diagram for explaining an operation of the third embodiment;

FIG. 16 is a perspective view illustrating a structure of each electrode at a distal end side of a high-frequency probe according to a fourth embodiment of the present invention;

FIG. 17 is a perspective view illustrating a structure of each electrode at a distal end side of a high-frequency probe according to a first modification of the fourth embodiment;

FIG. 18 is a perspective view illustrating a structure of each electrode at a distal end side of a high-frequency probe according to a second modification of the fourth embodiment; and

FIG. 19 is a diagram illustrating a relationship, for example, between a known electrocardiographic waveform and an intracardiac atrial pressure.

### DETAILED DESCRIPTION OF THE PREFERRED EMBODIMENTS

With reference to the drawings, hereinafter will be described some embodiments of the present invention.

### First Embodiment

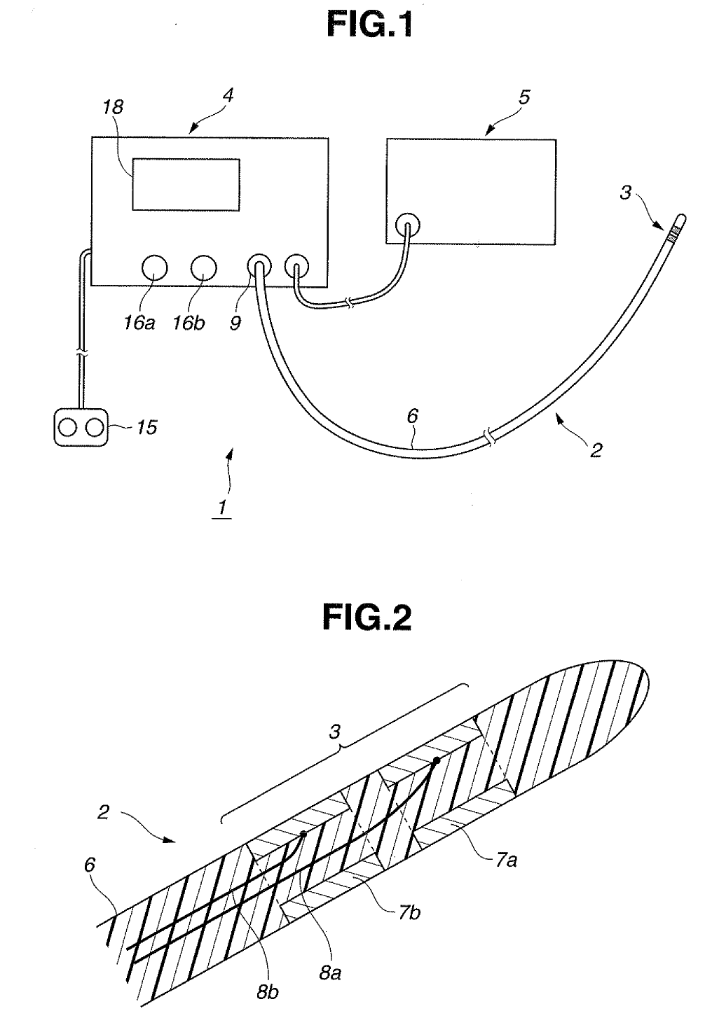

As shown in FIG. 1, a high-frequency surgical device 1 of a first embodiment of the present invention includes: a high-frequency probe 2 serving as a high-frequency treatment tool to perform high-frequency surgery for a region to be treated of a patient; and a high-frequency power unit 4 to which a rear end of the high-frequency probe 2 is detachably connected to supply high-frequency power to a treatment section 3 at a distal end of the high-frequency probe 2, for conducting high-frequency surgery. Biological information is inputted to the high-frequency power unit 4 from an electrocardiogram measuring device 5 which serves as biological information measuring means for measuring biological information associated with a region to be treated.

A high-frequency surgical system is formed by the high-frequency surgical device 1 and the electrocardiogram measuring device 5 as the biological information measuring means. Alternatively, the high-frequency surgical device 1 may be configured to include the electrocardiogram measuring device 5.

The high-frequency probe 2 is formed, for example, of an axial member 6 having a diameter which is small enough to be inserted into a blood vessel. As shown in FIG. 2, the axial member has the treatment section 3 at a distal end portion of the member, which is provided with two annular electrodes 7a and 7b as bipolar electrodes adjacently disposed in the longitudinal direction.

The axial member 6 is formed of a material, such as fluorinated resin, having a proper degree of flexibility and good electrical insulation properties as well.

The electrodes 7a and 7b are each formed into an annular shape with a conductive material, such as gold or platinum, so as to be externally exposed at a distal end portion of the axial member 6 having electrical insulation properties.

Also, leads 8a and 8b are inserted through the axial member 6, with distal ends of the leads being connected to the electrodes 7a and 7b, respectively, and with rear ends of the leads being connected to contact points of a connector 9 which is provided at a rear end of the axial member 6. The connector 9 is detachably connected to a connector receiver of the high-frequency power unit 4.

Alternatively to the structure mentioned above, the axial member 6 and the leads 8a and 8b inserted therethrough may be separated from each other, on the rear end side of the axial member 6. Alternatively, the axial member 6 may have a tubular structure with a hollow portion being provided therein.

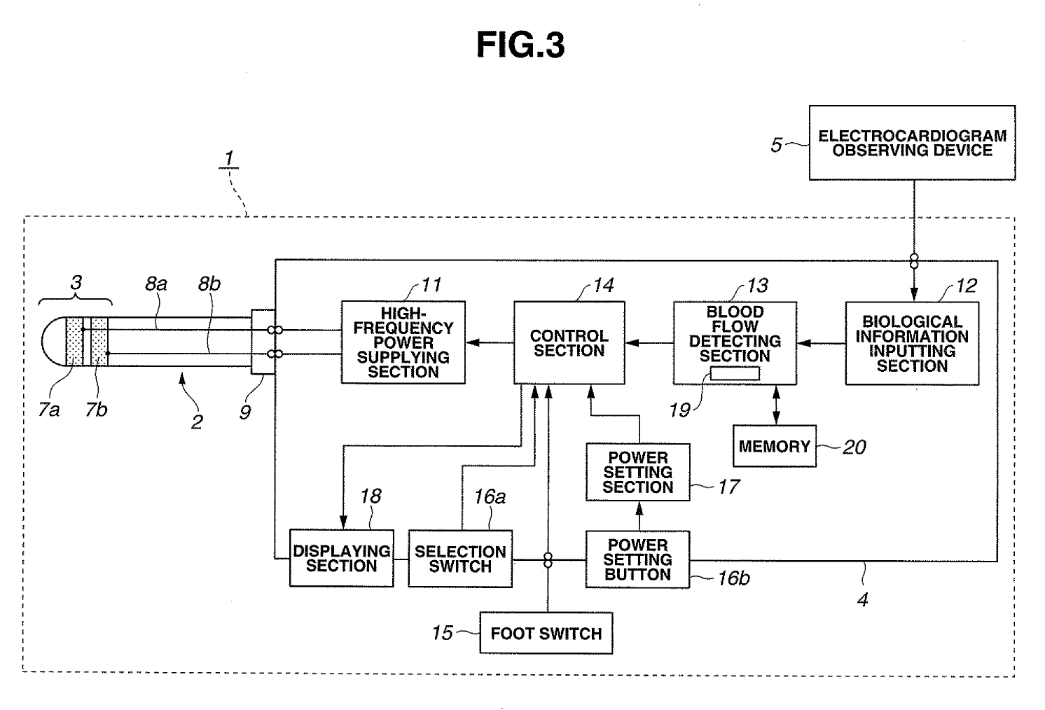

FIG. 3 shows an internal configuration of an electrical system, or chiefly, the high-frequency power unit 4, in the high-frequency surgical device 1. As shown in FIG. 3, the high-frequency power unit 4 includes: a high-frequency power supplying section 11 for supplying high-frequency power to living tissue to be treated through the electrodes 7a and 7b; a biological information inputting section 12 for inputting electrocardiographic waveform signals as measurement signals of biological information inputted from the electrocardiogram measuring device 5; a blood flow detecting section 13 for detecting intracardiac blood flow, or, in particular, timing of a period when blood flow velocity is high, in the form of blood flow information, based on the electrocardiographic waveform signals inputted from the biological information inputting section 12; and a control section 14 for controlling power to be applied (supplied) to the electrodes 7a and 7b, based the blood flow information of the period when the intracardiac blood flow velocity is high, which has been derived from the blood flow detecting section 13.

The high-frequency power unit 4 is provided with a foot switch 15 for an operator to control application (supply)/stoppage of the high-frequency power. The control section 14 effects control for applying/stopping the high-frequency power of the high-frequency power supplying section 11, in response to operational signals from the foot switch 15.

In this regard, in a normal control mode, the control section 14 will apply/stop the high-frequency power of the high-frequency power supplying section 11 in response to the operational signals from the foot switch 15. However, in a control mode of the present embodiment, in which treatment with the high-frequency power is given based on the input of the biological information, the control for applying/stopping the high-frequency power is effected in synchronization with detection signals (or measurement signals) in the electrocardiographic waveform signals derived from the blood flow detecting section 13.

Thus, the high-frequency power unit 4 is provided, at its front panel, with a selection switch 16a so that the operator can select such control modes.

Also, the high-frequency power unit 4 is provided, at its front panel, with a power setting button 16b so that the operator can set and instruct a high-frequency power value. A signal for setting and instructing a value with the power setting button 16b is inputted to the control section 14 through a power setting section 17. The control section 14 controls the high-frequency power supplying section 11 so that the high-frequency power supplying section 11 can output high-frequency power with the high-frequency power value that has been set at the power setting section 17.

Further, the high-frequency power unit 4 is provided, at its front panel, with a displaying section 18 for displaying various types of information under the control of the control section 14.

The blood flow detecting section 13 is provided, in advance, with information on the results of an analysis conducted for the electrocardiographic waveform signals. The analysis, in particular, is on the blood flow velocity in the vicinity of the PFO in a heart, which is a region to be treated with the high-frequency power (high-frequency current). The information on the analytical results is stored in a memory 20, for example, connected to the blood flow detecting section 13.

For example, the memory 20 stores information on the analytical results concerning “electrocardiographic waveform—blood flow velocity” indicating which portion of a signal waveform in the electrocardiographic waveform corresponds to the period of high blood flow velocity.

The information mentioned above corresponds to information on each period from an R wave to a T wave in an electrocardiographic waveform that will be described later. The blood flow detecting section 13 is provided with a specific electrocardiographic waveform detector 19, which detects a specific portion in the electrocardiographic waveform, corresponding to (start and end timings of) each period of high blood flow velocity, upon input of the electrocardiographic waveform signals from the electrocardiogram measuring device 5.

More specifically, the specific electrocardiographic waveform detector 19 includes: a first specific electrocardiographic waveform detector for detecting a first specific electrocardiographic waveform (timing thereof) at which a level of blood flow velocity rises to a first predetermined value or more; and a second specific electrocardiographic waveform detector for detecting a second specific electrocardiographic waveform (timing thereof) at which the level of the blood flow velocity that has risen to the first predetermined value or more drops to a level of a second predetermined value or less.

The specific electrocardiographic waveform detector 19 configuring the blood flow detecting section 13 outputs to the control section 14 a first detection signal that has detected the first specific electrocardiographic waveform and a second detection signal that has detected the second specific electrocardiographic waveform, as blood flow information on high blood flow velocity.

Particularly, the specific electrocardiographic waveform detector 19 has a peak detection circuit for detecting the R wave, which serves as the first specific electrocardiographic waveform detector. The peak detection circuit also serves as a peak detection circuit for detecting the T wave.

In synchronization with the first and second detection signals outputted from the blood flow detecting section 13, the control section 14 temporally controls the timings of supply and stoppage of the high-frequency power from the high-frequency power supplying section 11, for the electrodes 7a and 7b. In this way, the control section 14 temporally controls supply and stoppage of the high-frequency power in synchronization with the specific timings in the electrocardiographic waveform signals corresponding to the period of high blood flow velocity.

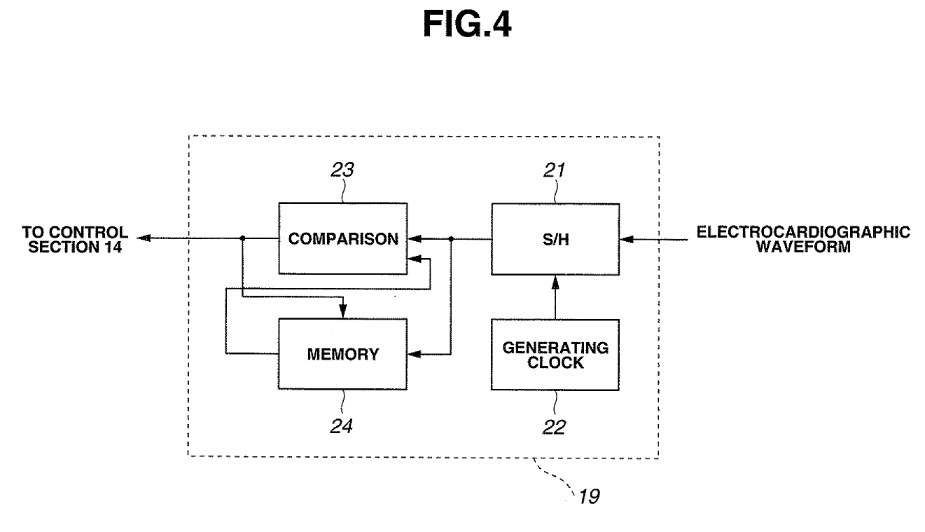

FIG. 4 shows an example of a configuration of the peak detection circuit configuring the specific electrocardiographic waveform detector 19.

The peak detection circuit, to which the electrocardiographic waveform signals are inputted, has a sample-hold (abbreviated as “S/H”) circuit 21 for sampling and holding input signals. The S/H circuit 21 samples and holds the input signals in synchronization with clocks from a clock generation circuit 22 to output the signals that have been sampled and held to a comparator circuit 23 and a memory 24.

The comparator circuit 23 compares an output signal from the S/H circuit 21 with a signal read out and outputted from the memory 24 as a reference signal, to thereby output a signal indicative of the comparison results. When the output signal from the S/H circuit 21 is larger than the reference signal, the comparator circuit 23 effects control for overwriting the reference signal in the memory 24 with the output signal (so as to renew the previous signal).

In this way, as a result of comparison, the comparator circuit 23 outputs the first detection signal that has detected a peak value to the control section 14, at a comparison-result timing when the reference signal of the memory 24 is larger than the output signal from the S/H circuit 21.

Upon input of the first detection signal, the control section 14 starts supplying the high-frequency power to the electrodes 7a and 7b by controlling the high-frequency power supplying section 11, as will be described later.

Also, on or after the timing of inputting an S wave of the electrocardiographic waveform from the timing when the first detection signal has been outputted, the peak detection circuit starts a peak-detecting operation for detecting the T wave. Then, upon detection of the T wave, the peak detection circuit outputs the second detection signal to the control section 14.

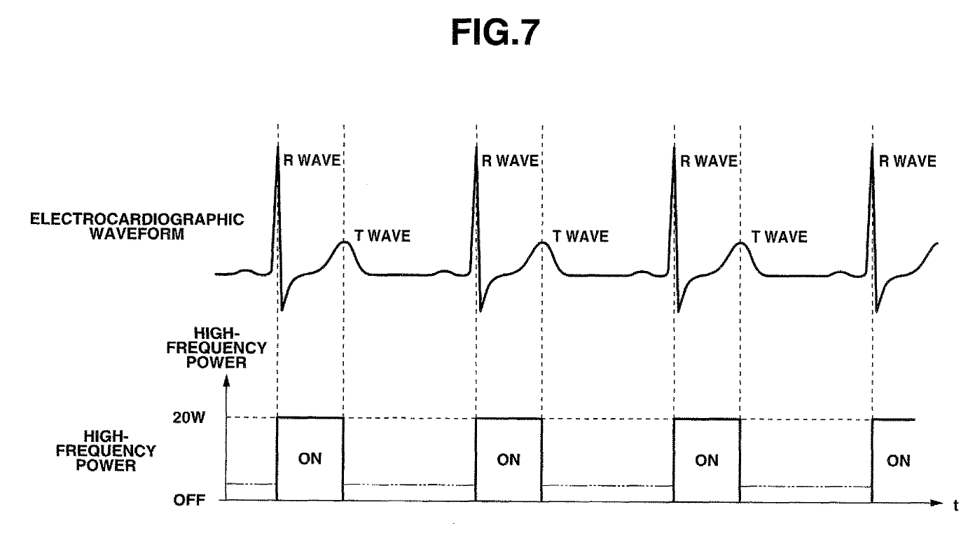

With the input of the second detection signal, the control section 14 stops supply of the high-frequency power by the high-frequency power supplying section 11 (specific operations will be explained later as shown in FIG. 7).

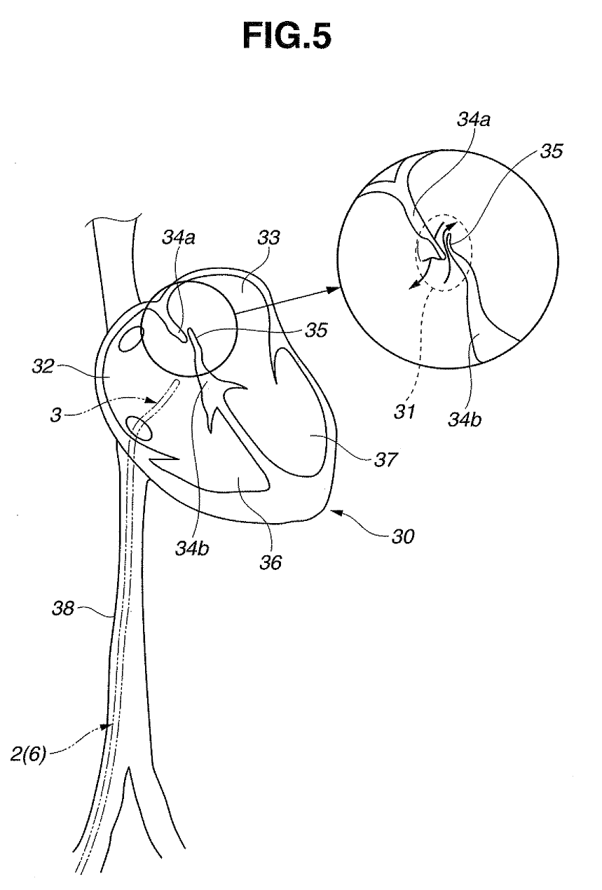

The present embodiment is to give treatment to the PFO in a heart using the high-frequency power, and thus an explanation hereinafter is given on the PFO in a heart with reference to FIG. 5.

As shown in FIG. 5, a PFO 31 is present in a portion of atrial septa 34a and 34b that separate a right atrium 32 from a left atrium 33 in a heart 30. FIG. 5 shows a state where a valve 35 of oval foramen (hereinafter referred to as “oval foramen valve 35”) is open being apart from the atrial septum 34a. Blood flows around the PFO 31 as shown by the arrows in the figure.

Also, a right ventricle 36 and a left ventricle 37 are present below the right atrium 32 and the left atrium 33, respectively.

In the present embodiment, in the case where treatment is given for occluding the PFO 31 using the high-frequency probe 2, the high-frequency probe 2 is inserted, for example, into an inferior vena cava 38 communicating with the right atrium 32, as shown by a dash-dot-dot line.

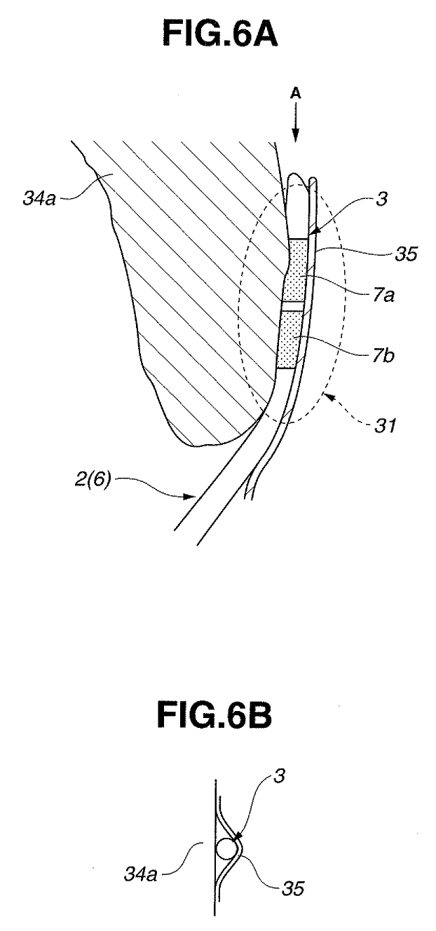

Then, a distal end side of the high-frequency probe 2 is inserted into the right atrium 32 from an opening communicating with the right atrium 32 to set the treatment section 3 provided at the distal end side of the high-frequency probe 2 to the PFO 31, as shown in FIG. 6A.

FIG. 6B shows the state of FIG. 6A as viewed from the distal end side of the high-frequency probe 2, i.e. from a direction of reference A.

As shown in FIGS. 6A and 6B, the electrodes 7a and 7b provided in the treatment section 3 at the distal end side of the high-frequency probe 2, are located in the PFO 31, i.e. located between the atrial septum 34a and the oval foramen valve 35.

After positioning and setting in the PFO 35 the treatment section 3 provided at the distal end side of the high-frequency probe 2 as shown in FIGS. 6A and 6B, the treatment for occluding the PFO 31 is given by supplying the high-frequency power to the electrodes 7a and 7b of the treatment section 3.

Thus, the electrodes 7a and 7b are located between the atrial septum 34a and the oval foramen valve 35, so that, when the high-frequency energy is supplied to the electrodes 7a and 7b, an area where the electrodes 7a and 7b are in contact with blood will be small. In this way, it is ensured that a treatment of high-frequency cautery using high-frequency energy for occluding the PFO 31 can be effectively given.

The timing for actually supplying the high-frequency power to the electrodes 7a and 7b of the treatment section 3 is controlled by the control section 14.

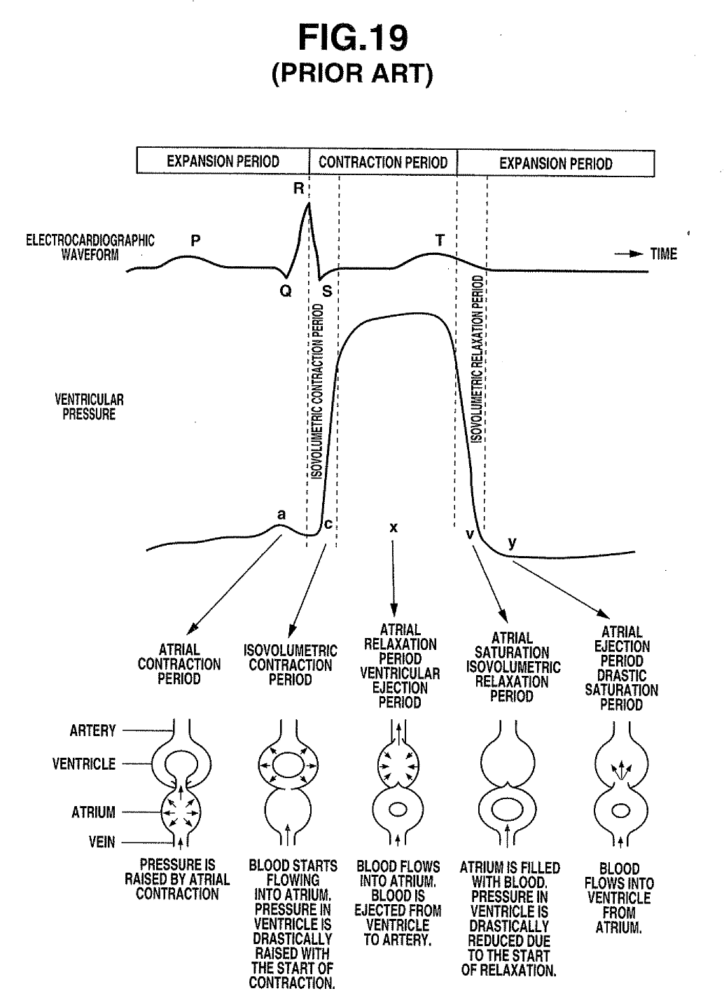

In this regard, referring now to FIG. 19 illustrating prior art, an explanation hereinafter is given on the electrocardiographic waveform outputted from the electrocardiogram measuring device 5, as well as actions taken by the ventricles, the atria, the vein and the like in the heart 30.

As shown in FIG. 19, one heartbeat in an electrocardiographic waveform includes from P wave, Q wave, R wave, S wave and up to T wave, with alternate repetition of an expansion period and a contraction period. Among the waves, prominent Q, R and S waves are collectively called a QRS wave. Ventricular pressure is high between an isovolumetric contraction period and an isovolumetric relaxation period and is low in other periods. FIG. 19 schematically shows at its bottom, corresponding to the changes of the venticular pressure, an overview of pressure change, blood flow, and the like.

The blood flow detecting section 13 of the present embodiment carries out in advance an analysis of blood flow velocity in the atria based on the electrocardiographic waveform. The acquired analytical results show that, as shown in FIG. 7, waveform sharply rises up to form the peak, or the R wave (i.e. the R wave that is the timing for starting contraction of the ventricles), and falls down to form the S wave, and that a period after the S wave up to a small peak, or the T wave (i.e. the T wave that is the timing for starting expansion of the ventricles), is a period during which blood flows into the atria to increase the blood flow velocity in the atria.

On the basis of the analytical results, the specific electrocardiographic waveform detector 19 of the blood detecting section 13 detects the R waves and the T waves in the electrocardiographic waveform received from the biological information inputting section 12.

Then, as shown in FIG. 7, the control section 14 effects control so that the high-frequency power of a preset power value (20 W in the example of FIG. 7) can be supplied to the side of the electrodes 7a and 7b only during the periods of high blood flow velocity, or only when blood flows rapidly, whereby the treatment of high-frequency cautery is performed with the high-frequency energy.

Thus, the treatment of flowing high-frequency power only during the periods of high blood flow velocity can prevent blood clots from being formed and thus can contribute to effectively giving the treatment of occlusion to the PFO 31.

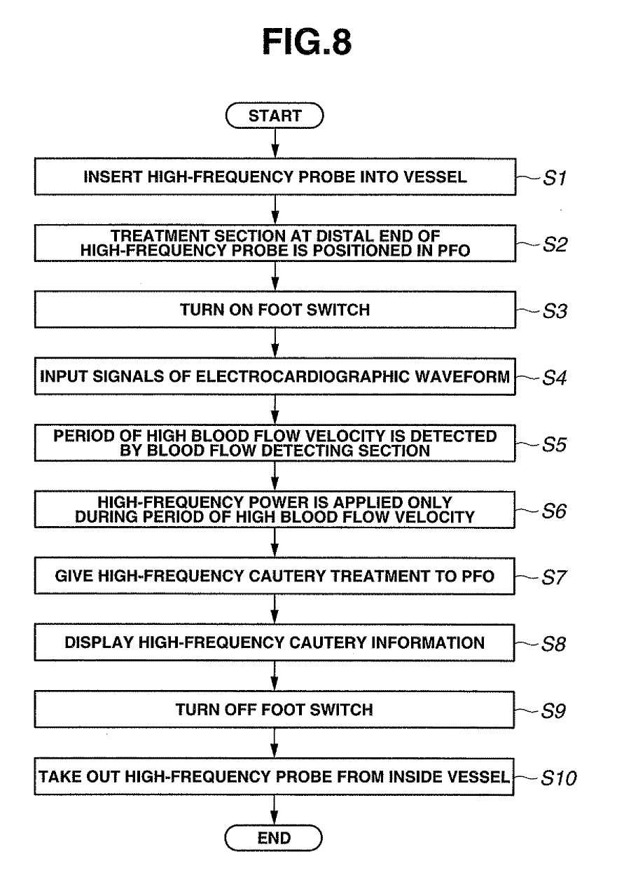

Referring now to FIG. 8, hereinafter is explained a procedure for giving the treatment of occlusion to the PFO 31, using the high-frequency surgical device 1 as described above.

First, the high-frequency apparatus 1 is set by the operator as shown in FIG. 1. Also, electrodes (not shown) of the electrocardiogram measuring device 5 are attached to a patient to be operated to obtain electrocardiographic waveform.

Then, when the high-frequency surgical device 1 and the electrocardiogram measuring device 5 are turned on by the operator, both of the devices are brought into an operational state.

At step S1, the high-frequency probe 2 is inserted into a blood vessel of the patient by the operator. For example, as shown in FIG. 5, the high-frequency probe 2 is inserted into the vessel of the inferior vena cava 38 of the patient by the operator.

Then, at the subsequent step S2, the distal end side of the high-frequency probe 2 is inserted into the right atrium 32 of the heart 30 by the operator to locate and set the treatment section 3 in the PFO 31 which is positioned deeper than the right atrium. For example, the treatment section 3 is set by the operator as shown in FIGS. 6A and 6B.

After positioning the treatment section 3 in the PFO 31 that is the region to be treated, the foot switch 15 is depressed (turned on) by the operator, at step S3.

Then, the control section 14 starts controlling timing for actually applying the high-frequency power from the high-frequency power supplying section 11 to the side of the electrodes 7a and 7b, according to the detection signals from the blood flow detecting section 13. At step S4, the electrocardiographic waveform signals are inputted to the blood flow detecting section 13 from the electrocardiogram measuring device 5 through the biological information inputting section 12.

At step S5, the blood flow detecting section 13 starts an operation for detecting the periods of high blood flow velocity from the electrocardiographic waveform, based on the results of analysis on the waveform. After detecting the periods of high blood flow velocity, the detection signals are outputted to the control section 14. Specifically, the specific electrocardiographic waveform detector 19 of the blood flow detecting section 13 detects the R waves and the T waves as shown in FIG. 7 and outputs the detection signals to the control section 14.

At step S6, the control section 14 effects control so that the high-frequency power from the high-frequency power supplying section 11 can be applied to the side of the electrodes 7a and 7b only during the periods of high blood flow velocity, in response to the detection signals inputted during the periods of high blood flow velocity.

Then, at step S7, the treatment of high-frequency cautery is given to the PFO 31 with the high-frequency energy only during the periods of high blood flow velocity.

Being given the treatment of high-frequency cautery with the high-frequency energy, a region of the PFO 31 around the electrodes 7a and 7b is heated and damaged for remedy. It should be appreciated that, after the high-frequency cautery treatment, the damaged living tissue heals.

In this way, the high-frequency cautery treatment is given only during the periods of high blood flow velocity. As a result, the blood heated by the high-frequency cautery will move in a short time away from the positions where the blood has been heated, due to the high blood flow velocity.

In other words, although the blood is heated sometime during the high-frequency cautery, the heat energy is diffused in a short time to sufficiently suppress temperature rise in the heated blood, whereby the blood can be effectively prevented from being formed into clots. On the other hand, standing stationarily, the living tissue of the PFO 31 to be treated can store therein the heat of the high-frequency cautery without the heat diffusion. Accordingly, the living tissue is allowed to be damaged by the high-frequency cautery.

At the subsequent step S8, the control section 14 indicates, on the displaying section 18, the high-frequency cautery information on the periods, for example, when the high-frequency cautery is actually performed. In the case where a power value and periods for performing the high-frequency cautery have been set in advance, the control section 14 indicates, on the displaying section 18, the periods of the high-frequency cautery, a cumulative period of the high-frequency cautery, and the like. With the indication of the information, the operator can be notified of the progress in the high-frequency cautery treatment.

When the predetermined period of treatment with the predetermined power value has expired, the control section 14 then effects control so that the displaying section 18 can indicate the fact that the high-frequency cautery treatment has been finished. With the finishing indication, the operator can turn off the foot switch 15, at step S9. Alternatively, it may be so configured that the turning off of the foot switch 15 is performed by the control section 14.

At the subsequent step S10, the operator can take out the high-frequency probe 2 from the vessel to end the treatment of occlusion for the PFO 31.

As described above, the living tissue of the PFO 31 is heated and damaged with the application of the high-frequency power. After the high-frequency cautery treatment, however, the damaged living tissue heals. Usually, the atrial septum 34a and the oval foramen valve 35 are naturally pressed and in contact with each other, and thus the atrium septum 34a and the oval foramen valve 35 are fused during the healing to achieve natural occlusion. In this way, the occlusion of the PFO 31 is completed.

As described above, according to the present embodiment, the R waves and the T waves of high blood flow velocity are detected from the electrocardiographic waveform to enable application of the high-frequency power to the PFO 31 only during the periods of high blood flow velocity. In this way, high-frequency cautery treatment can be given for occluding the PFO. At the same time, blood temperature rise can be suppressed in the region around the PFO 31 during the cautery treatment to prevent the formation of blood clots.

In the explanation provided above, current has been supplied only during the periods of high blood flow velocity, that is, control has been effected so that the high-frequency power can be intermittently supplied. Alternatively, however, the high-frequency power can be increased or decreased according to the blood flow velocity.

For example, when the blood flow velocity is low, or when the blood flows slowly, control may be so effected that the power will be decreased, an example of which is indicated in FIG. 7 by a dash-dot-dot line. In FIG. 7, the dash-dot-dot line exemplifies a control in which a high-frequency power value is made smaller in the case where the blood flow velocity is low, than in the case where the blood flow velocity is high. With such a control, the advantages substantially similar to those described above can be enjoyed.

### Second Embodiment

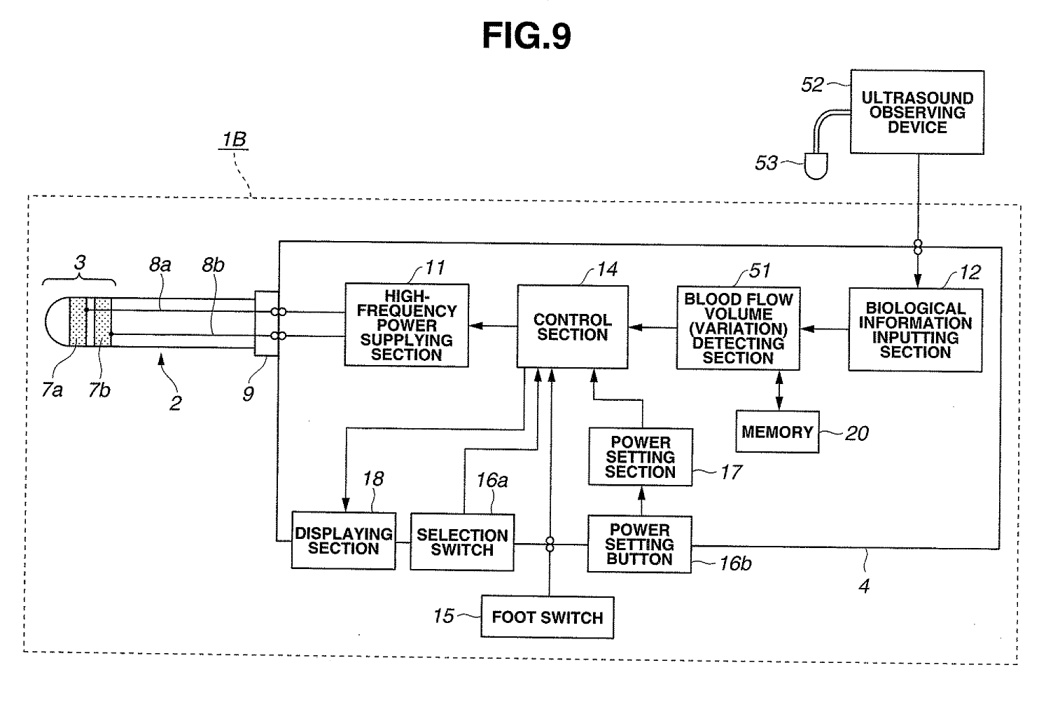

With reference to FIGS. 9 to 11, hereinafter will be described a second embodiment of the present invention.

FIG. 9 is a schematic diagram illustrating a high-frequency surgical device 1B according to the present embodiment. In the high-frequency surgical device 1 shown in FIG. 3, the high-frequency surgical device 1B includes a blood flow volume (variation) detecting section 51 for detecting blood flow volume or variation of blood flow, replacing the blood flow detecting section 13. Hereinafter, the blood flow volume (variation) detecting section 51 is referred to just as “blood flow volume detecting section 51”. Also, in the present embodiment, ultrasound information obtained from an ultrasound probe 53 of an ultrasound observing device 52 is inputted as the biological information, instead of the electrocardiographic waveform signals from the electrocardiogram measuring device 5.

The ultrasound observing device 52 includes: an ultrasound displaying mode of a color Doppler mode utilizing the Doppler phenomenon; and ultrasound information outputting means.

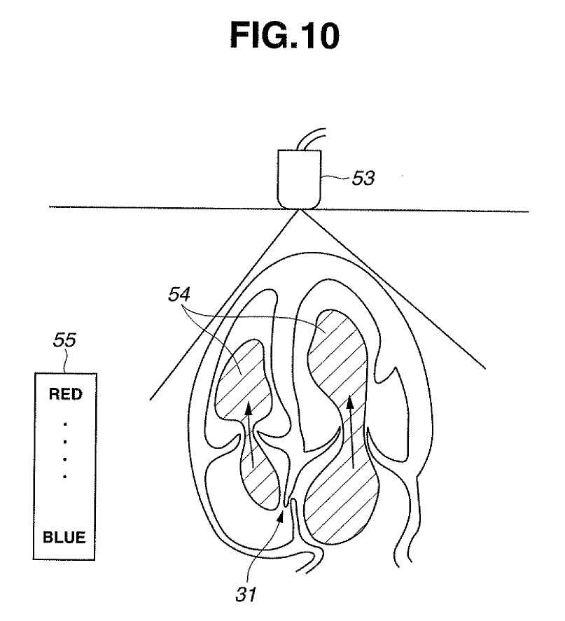

FIG. 10 is a schematic diagram in which the ultrasound probe 53 is brought into contact with a chest of a patient to be operated, with an indication of an intracardiac blood flow volume variation in the color Doppler mode. In this case, when blood flows from the right atrium to the right ventricle and from the left atrium to the left ventricle, as shown by arrows (from bottom to top in FIG. 10), for example, that is, when blood flows toward the ultrasound probe 53, blood-flowing portions 54 (shaded areas) are displayed in red.

On the left side, for example, of FIG. 10, a color gauge 55 is indicated. In the gauge 55, portions approaching the side of the ultrasound probe 53 that is a sound source, are indicated in red, and portions staying away from the ultrasound probe 53 are indicated in blue. The blood flow volume varies with the level and the size of the red color.

The blood flow volume detecting section 51 of the present embodiment analyzes the intracardiac blood flow volume or variation of blood flow volume, based on the ultrasound information which is obtained with the use of the ultrasound probe 53, and stores the information on the analytical results in the memory 20.

Then, based on the information on the analytical results, in the case where the ultrasound signals of the color Doppler mode are actually inputted in the form of the ultrasound information from the ultrasound observing device 52, the blood flow detecting section 51 outputs detection signals to the control section 14 as blood flow information informing of large blood flow volume, at start and end timings of each period when the blood flow volume in the vicinity of the PFO 31, which is located at the boundary of the right and left atria, becomes equal to or larger than a preset value.

For example, the blood flow volume detecting section 51 outputs to the control section 14 the detection signals at the timings of the ultrasound signals corresponding to the start and end of each period which corresponds to the blood flow state as shown in FIG. 10.

The control section 14 controls the supply (application) and stoppage of the high-frequency power of the high-frequency power supplying section 11 in synchronization with the detection signals inputted at the start and end timings of each period when the blood flow volume becomes large. Sometimes, there may be a time lag from when the ultrasound observing device 52 has actually produced an ultrasound signal of the color Doppler mode up to when the ultrasound signal is outputted to the biological information inputting section 12. In such a case, the blood flow volume detecting section 51 carries out temporal adjustment by, for example, detecting an ultrasound signal portion at the timing shifted behind by the time equivalent to the time lag. The remaining configuration is the same as the first embodiment.

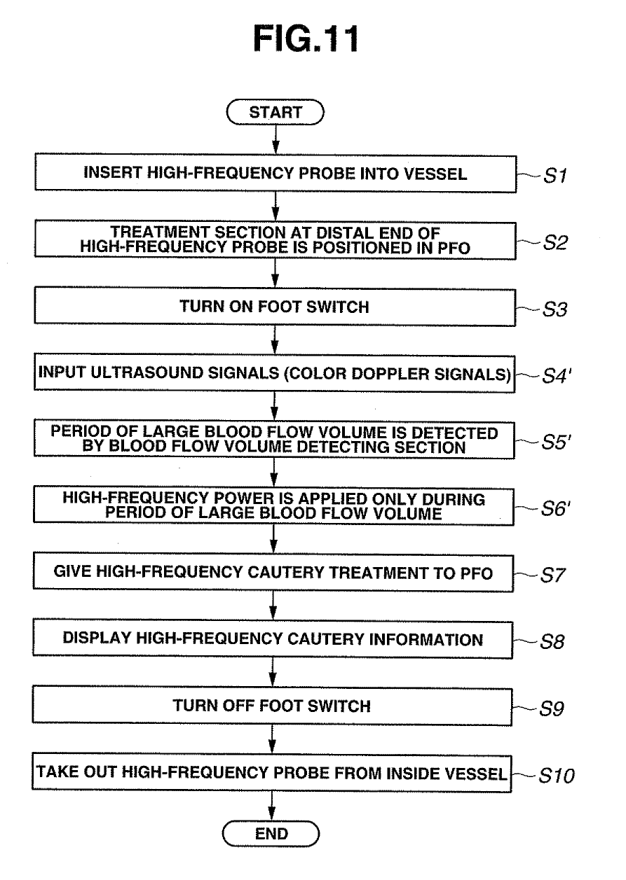

FIG. 11 shows an example of a procedure of a method for giving a treatment of occlusion to the PFO 31, according to the present embodiment. The steps shown in FIG. 11 have contents which are partially changed from those shown in FIG. 8 that has been used for explaining the first embodiment.

Specifically, steps S1 to S3 in FIG. 11 are the same as those in FIG. 8. However, the ultrasound observing device 52 is used instead of the electrocardiogram measuring device 5. At step S4′ subsequent to step S3, the ultrasound signals (color Doppler signals) are inputted from the ultrasound observing device 52 to the blood flow volume detecting section 51 through the biological information inputting section 12.

Then, at step S5′, the blood flow detecting section 51 detects periods when the blood flow volume is large, the resultant of which is outputted to the control section 14.

At the subsequent step S6′, the high-frequency power supplying section 11 applies the high-frequency power to the electrodes 7a and 7b only during the periods of large blood flow volume, under the control of the control section 14. The steps from step S6′ onward, i.e. steps S7 to S10 are the same as those of FIG. 8.

In this way, in the present embodiment, the high-frequency power is supplied to the electrodes 7a and 7b at the distal end of the high-frequency probe 2 only during the periods when the blood flow volume becomes large in the vicinity of the PFO to thereby perform the high-frequency cautery treatment. Specifically, the high-frequency cautery treatment is given during the periods of large blood flow volume to suppress the formation of blood clots and thus to smoothly give the treatment to the PFO.

The description provided above has been given on the case where ultrasound information is used in the color Doppler mode of the ultrasound observing device 52.

The ultrasound observing device 52 has another mode different from the color Doppler mode, that is, a mode for indicating or outputting variation of blood flow at a specific position, with the conversion into a graph (Doppler mode).

Thus, the configuration may be such that the Doppler mode is selected to measure the intra-atrial variation of blood flow, and then, only when the blood is in the process of increasing up to a value equal to or more than a preset value, to apply the high-frequency power.

Similar to the first embodiment, the configuration mentioned above enables application of the high-frequency power to the electrodes 7a and 7b only during the periods when blood is in the process of increasing to thereby effectively give the treatment of occlusion to the PFO 31 by preventing the formation of blood clots.

Thus, according to the present embodiment, the high-frequency power can be applied to the electrodes 7a and 7b only during the periods when the blood flow volume around the PFO 31 is increased. Thus, due to the increase of the blood flow volume, temperature rise of the blood around the PFO 31 can be prevented, whereby the treatment can be given in the state where formation of blood clots is prevented.

### Third Embodiment

Referring now to FIGS. 12 to 15, hereinafter is described a third embodiment of the present invention. FIG. 12 shows a high-frequency probe 2C used for a high-frequency surgical device 1C according to the present embodiment. FIG. 12 particularly shows the treatment section 3 and the vicinity thereof at the distal end side of the probe 2C. In the high-frequency probe 2 shown in FIG. 1 or 2, the high-frequency probe 2C is provided with a fluid ejection port 61 in the vicinity of the treatment section 3 at the distal end side of the probe.

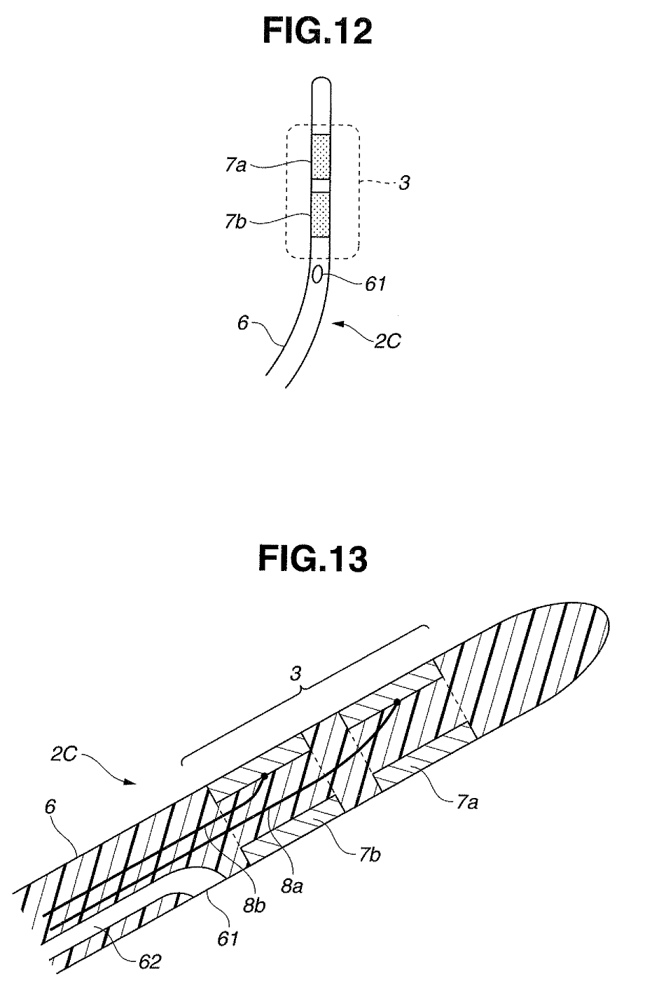

FIG. 13 is an enlarged cross-sectional view of the distal end side of the probe shown in FIG. 12. As shown in FIG. 13, the fluid ejection port 61 is in communication with a fluid lumen 62 provided along a longitudinal direction of the axial member 6.

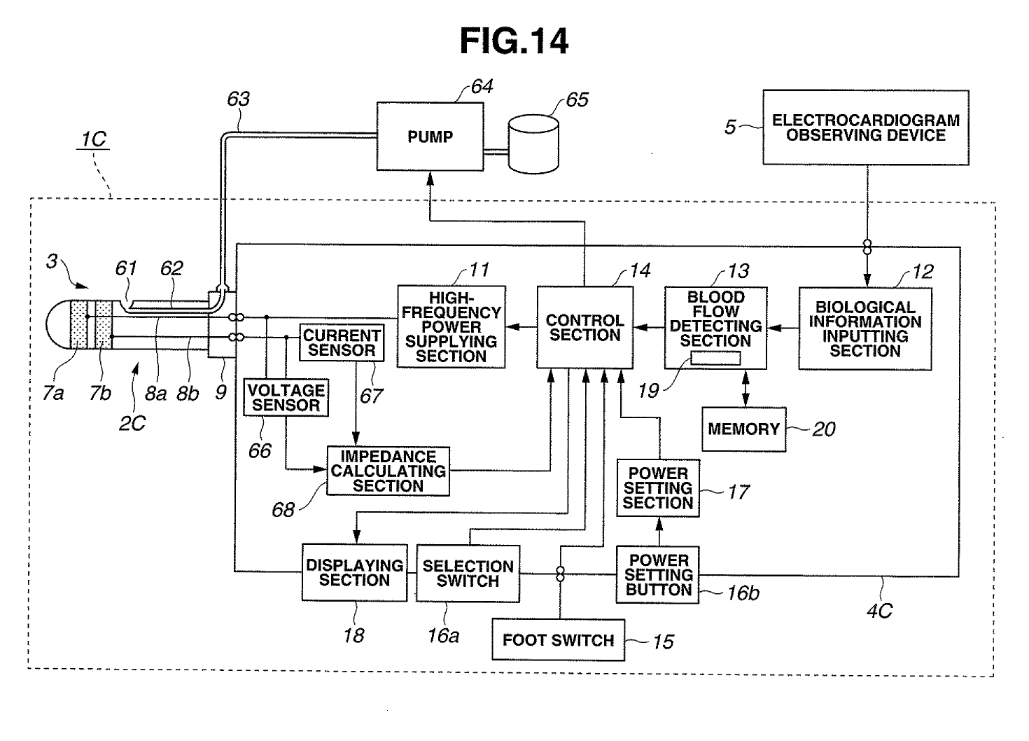

As shown in FIG. 14, a rear end of the fluid lumen 62 is connected to a pump 64 serving as fluid delivering means for delivering fluid, through a fluid delivery tube 63 which is connected to the connector 9. The pump 64 is connected with a reservoir 65 which stores fluids, such as normal saline and contrast medium agent, which are innoxious to human beings.

The remaining configuration of the high-frequency probe 2C is the same as the configuration explained in the first embodiment.

FIG. 14 is a schematic diagram illustrating the high-frequency surgical device 1C according to the present embodiment. The high-frequency surgical device 1C includes the high-frequency probe 2C and a high-frequency power unit 4C.

In the high-frequency power unit 4 shown in FIG. 3, the high-frequency power unit 4C includes: a voltage sensor 66 for measuring high-frequency voltage supplied (applied) to the side of the electrodes 7a and 7b from the high-frequency power supplying section 11; a current sensor 67 for measuring high-frequency current supplied to the side of the electrodes 7a and 7b from the high-frequency power supplying section 11; and an impedance calculating section 68 for calculating (or measuring) impedance from the measured high-frequency voltage and high-frequency current.

Information on the impedance calculated by the impedance calculating section 68 is transferred to the control section 14. The control section 14 can then control the value of the high-frequency power supplied to the side of the electrodes 7a and 7b from the high-frequency power supplying section 11, based on the information on the impedance.

It should be appreciated that the impedance calculating section 68 may calculate (measure) a value of resistance (resistance).

The control section 14 has a function of controlling the operation of actuating and stopping the pump 64. The remaining configuration is the same as that explained in the first embodiment. The present embodiment has been exemplified as a configuration applied to the first embodiment, but may also be applied to the second embodiment.

Similar to the first embodiment, the present embodiment enables application of the high-frequency power to the side of the PFO, as well as the ejection of fluid to the vicinity of the PFO from the fluid ejection port 61 by actuating the pump 64, upon the application of the high-frequency power.

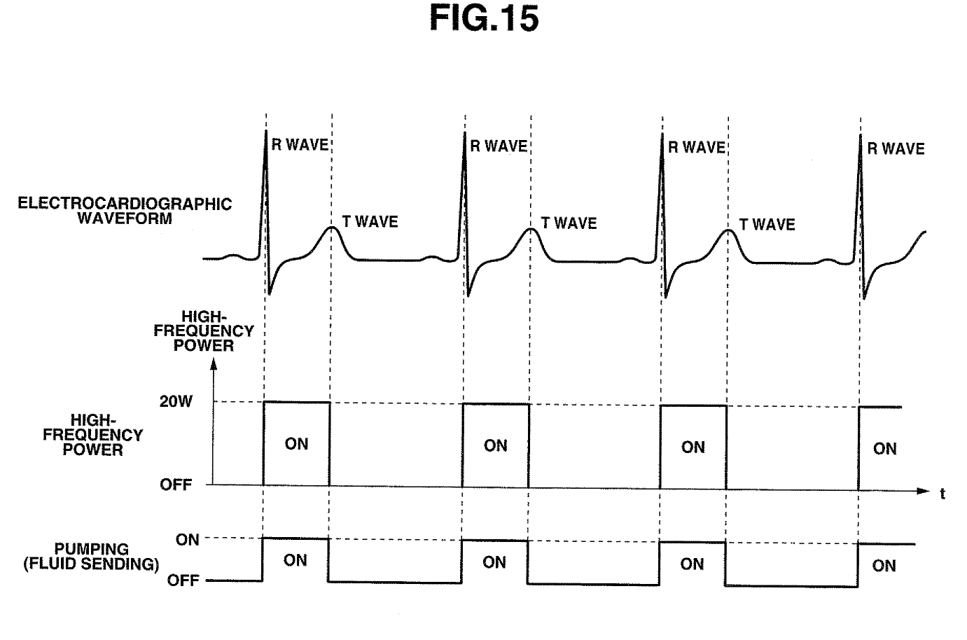

The operation in this case is shown in a timing diagram of FIG. 15. In the timing diagram of FIG. 7, FIG. 15 shows an additional timing diagram of a fluid delivery operation of the pump 64.

As shown in FIG. 15, the operation of fluid delivery is performed synchronizing with the supply (ON) of the high-frequency power. The present embodiment is adapted to additionally deliver fluid in synchronization with the supply of the high-frequency power in the first embodiment. As a result, temperature of the blood around the PFO can be further reduced to thereby further prevent formation of blood clots than in the first embodiment.

It is considered that the timing of applying the high-frequency power and for ejecting fluid may also be realized as follows.

For example, it may be so configured that the fluid is ejected (delivered) after expiration of a predetermined period from the supply of the high-frequency power.

Also, the method for controlling a flow rate of the fluid to be ejected may be realized as follows.

The flow rate of the fluid to be ejected may be controlled in proportion to the power value set by the operator. For example, when the set power value is large, the flow rate of ejection may be increased.

Alternatively, the flow rate of the fluid to be ejected may be controlled in accordance with an impedance value or a resistance value of the living tissue. For example, when the impedance or resistance value is small, the flow rate may be increased.

Alternatively, the flow rate of the fluid to be ejected may be controlled in proportion to the time of application of the high-frequency power. For example, when the time of application is to be long, the flow rate may be increased.

Alternatively, fluid in the reservoir may be cooled to further enhance the effects.

As described above, according to the present embodiment, fluid can be ejected around the PFO synchronizing with the application of the high-frequency power to the electrodes 7a and 7b. Accordingly, temperature rise of blood can be prevented around the PFO, and thus formation of blood clots can be prevented to effectively give the treatment of occlusion to the PFO.

### Fourth Embodiment

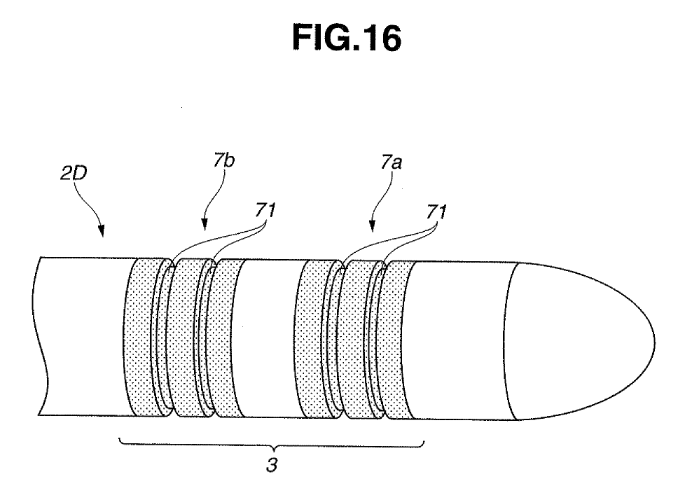

With reference to FIGS. 16 to 18, hereinafter is explained a fourth embodiment of the present invention. The present embodiment relates to a structure of a treatment section of a high-frequency probe. The present embodiment is applicable to any of the first to third embodiments.

FIG. 16 is an enlarged view of surfaces of electrodes located at a distal end side of a high-frequency probe 2D related to the fourth embodiment of the present invention.

In the present embodiment, the electrodes 7a and 7b of the first embodiment are formed into ring-shaped grooves 71 and 71, respectively, along the longitudinal direction, for example, of the electrodes so as to be perpendicular to the longitudinal direction, to thereby enlarge surface areas of the electrodes 7a and 7b. Accordingly, in the present embodiment, by enlarging the surface areas of the electrodes 7a and 7b, areas that will be in contact with living tissue to be treated can be enlarged to suppress the value of the high-frequency current that flows per unit area of the electrodes 7a and 7b.

In this way, the large surface areas of the electrodes 7a and 7b will reduce density of the high-frequency current around the electrodes 7a and 7b. Accordingly, temperature rise can be suppressed when the blood around the electrodes is heated and thus the blood can be prevented from being formed into clots.

Although FIG. 16 exemplifies two grooves 71 and 71, the number is not limited to two. Alternative to the example of FIG. 16, the following modified structures, for example, may be realized.

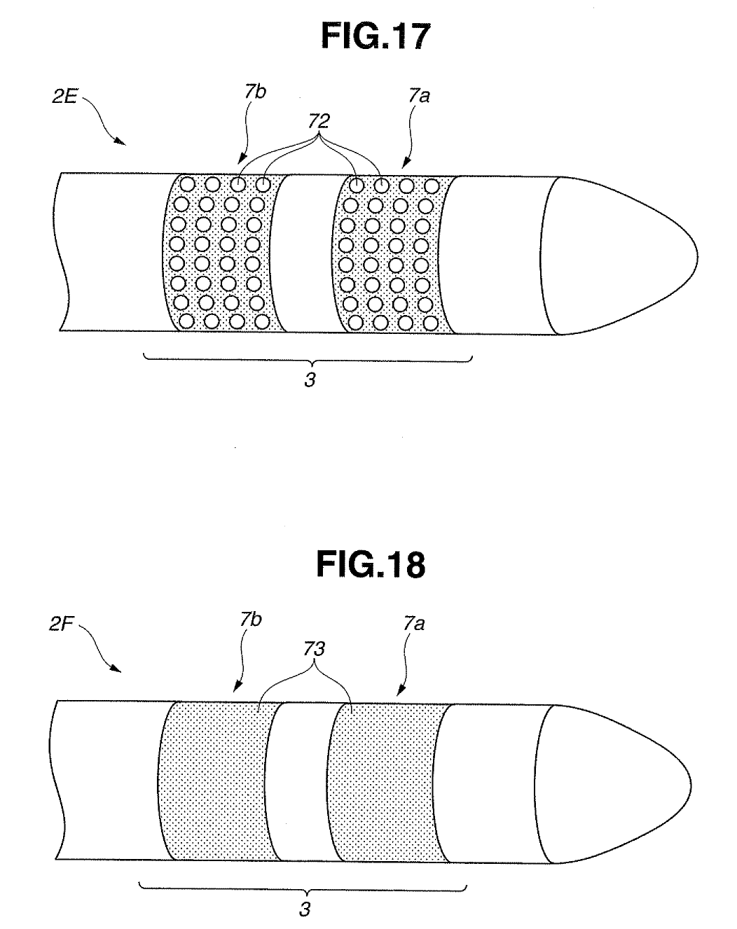

FIG. 17 is an enlarged view of surfaces of electrodes provided at a distal end side of a high-frequency probe 2E of a first modification.

In the high-frequency probe 2E, a number of circular recesses or projections 72 are provided on the surfaces of the electrodes 7a and 7b to enlarge the surface areas of the electrodes 7a and 7b. Alternatively, both of recesses and projections may be provided.

FIG. 18 is an enlarged view of surfaces of electrodes provided at a distal end side of a high-frequency probe 2F of a second modification.

In the high-frequency probe 2F, the surfaces of the electrodes 7a and 7b are roughened (like a surface of a file) to provide roughened portions 73 and thus to enlarge the surface areas of the electrodes 7a and 7b.

As described above, in applying the high-frequency power to the PFO through the electrodes 7a and 7b, the blood around the PFO is simultaneously heated.

Therefore, the large surface areas of the electrodes 7a and 7b for the reduction of the current density as shown in FIGS. 16 to 18, can prevent temperature rise of the blood near surface portions of the electrodes and can also prevent formation of blood clots.

As described above, according to the present embodiment temperature rise of the blood around the electrodes, as well as the formation of blood clots around the electrodes can be prevented in applying the high-frequency power to the electrodes, by enlarging the surface areas of the electrodes and reducing the density of the high-frequency current around the electrodes.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. A high-frequency surgical device comprising:
a treatment section provided with electrodes for supplying high-frequency power to living tissue of a patent foramen ovale;
a high-frequency power supplying section for supplying high-frequency power to living tissue around the electrodes through the electrodes;
a biological information inputting section for inputting biological information from outside;
a blood flow detecting section for detecting intracardiac blood flow information, based on the biological information inputted from the biological information inputting section; and
a control section for controlling high-frequency power to be supplied to the electrodes, based on the intracardiac blood flow information detected by the blood flow detecting section.

2. The high-frequency surgical device according to claim 1, wherein:
the biological information is made up of electrocardiographic waveform signals inputted from an electrocardiogram measuring device; and
the blood flow detecting section detects timings corresponding to a specific period of high blood flow velocity from the electrocardiographic waveform signal, as the blood flow information.

3. The high-frequency surgical device according to claim 1, wherein:
the biological information is made up of electrocardiographic waveform signals inputted from an electrocardiogram measuring device; and
the control section effects control by starting application of high-frequency power when an R wave is detected from the electrocardiographic waveform signals, and stopping application of high-frequency power when a T wave is detected from the electrocardiographic waveform signals.

4. The high-frequency surgical device according to claim 1, wherein:
the biological information is made up of electrocardiographic waveform signals inputted from an electrocardiogram measuring device, and
the blood detecting section detects timings corresponding to a period of high blood flow velocity in the electrocardiographic waveform signals, using a peak detection circuit for detecting a peak value of the electrocardiographic waveform signals.

5. The high-frequency surgical device according to claim 1, wherein:
the blood flow detecting section serves as a blood flow volume detecting section for detecting an intracardiac blood flow volume or variation of blood flow volume.

6. The high-frequency surgical device according to claim 1, wherein:
the biological information corresponds to information on intracardiac blood flow volume or variation of blood flow volume, the information being obtained by using ultrasound; and
the control section effects control by applying high-frequency power during a period when the blood flow volume is large.

7. The high-frequency surgical device according to claim 5, wherein:
the biological information corresponds to information on intracardiac blood flow volume or blood flow variation, the information being obtained by using ultrasound; and
the blood flow volume detecting section detects timings corresponding to the period when intracardiac blood flow volume is large by utilizing ultrasound Doppler phenomenon.

8. The high-frequency surgical device according to claim 5, wherein:
the biological information corresponds to information on intra-atrial blood flow variation, the information being obtained by using ultrasound; and
the control section applies high-frequency power during a period when the intra-atrial blood flow has increased to a level equal to or more than a predetermined value.

9. The high-frequency surgical device according to claim 1, further comprising:
a high-frequency treatment tool provided with the treatment section;
a fluid delivery lumen provided along a longitudinal direction of the high-frequency treatment tool to carry out fluid delivery;
a fluid ejection port provided in a vicinity of the treatment section to communicate with the fluid delivery lumen, for ejection of fluid; and
a fluid delivering device provided at a proximal end side of the fluid delivery lumen to deliver the fluid to the fluid ejection port through the fluid delivery lumen, wherein:
the control section controls start/stoppage of fluid delivering operation performed by the fluid delivering device, based on the blood flow information.

10. The high-frequency surgical device according to claim 1, wherein:
each of the electrodes has at least one of roughened, projected/recessed and grooved surfaces in order to reduce current density in the vicinity of the electrodes.

11. The high-frequency surgical device according to claim 1, wherein:
the treatment section having the electrodes is provided at a distal end side of an elongated high-frequency probe that can be inserted into a blood vessel of an inferior vena cava.

12. A high-frequency surgical method for giving a treatment of occlusion to a patent foramen ovale by supplying high-frequency power to living tissue of the patent foramen ovale through electrodes provided at a distal end side of a high-frequency probe, comprising:
a step of positioning and setting electrodes provided at a distal end side of a high-frequency probe, in living tissue of a patent foramen ovale to be treated;
a step of inputting biological information;
a step of detecting timings of the biological information corresponding to a period when blood flow velocity or blood flow volume is large, based on results of detection for the inputted biological information; and
a step of effecting control for supplying the high-frequency power to the electrodes during the period when blood flow velocity or blood flow volume is large, according to the step of detecting timing.

13. The high-frequency surgical method according to claim 12, wherein:
the biological information is made up of electrocardiographic waveform signals; and
the step of detecting timings comprises detecting timings corresponding to a specific period of high blood flow velocity from the electrocardiographic waveform signals.

14. The high-frequency surgical method according to claim 12, wherein:
the biological information is made up of electrocardiographic waveform signals; and
the step of effecting control comprises starting application of high-frequency power when an R wave is detected from the electrocardiographic waveform signals, the R wave corresponding to timing for starting ventricular contraction, and stopping application of high-frequency power when a T wave is detected from the electrocardiographic waveform signals, the T wave corresponding to timing for starting ventricular expansion.

15. The high-frequency surgical method according to claim 12, comprising:
a step of detecting the R wave from the electrocardiographic waveform signals; and
a step of detecting the T wave from the electrocardiographic waveform signals.

16. The high-frequency surgical method according to claim 12, wherein:
the biological information corresponds to information on intracardiac blood flow volume or blood flow variation, the information being obtained by using ultrasound; and
the step of detecting timings comprises detecting timings corresponding to a specific period when the blood flow volume is large.

17. The high-frequency surgical method according to claim 12, wherein:
the biological information corresponds to information on intracardiac blood flow volume or blood flow variation, the information being obtained by using ultrasound; and
the step of effecting control comprises effecting control for supplying the high-frequency power to a side of the electrodes during a specific period when blood flow volume is large.

18. The high-frequency surgical method according to claim 12, wherein:
the biological information corresponds to information on intra-atrial blood flow variation, the information being obtained by using ultrasound; and
the step of effecting control comprises effecting control for supplying the high-frequency power to a side of the electrodes during a period when the intra-atrial blood flow has increased to a level equal to or more than a predetermined value.

19. The high-frequency surgical method according to claim 12, wherein:
the step of setting electrodes comprises:
inserting the high-frequency probe into the blood vessel; and
locating two electrode portions between an atrial septum and a valve of oval foramen as living tissue that forms a patent foramen ovale, each of the electrode portions having a shape of a ring and provided at a distal end side of the high-frequency probe.

20. The high-frequency surgical method according to claim 12, wherein:
the step of effecting control comprises:
effecting control for supplying the high-frequency power to a side of the electrodes during a specific period when the blood flow velocity or blood flow volume is large; and
effecting control for reducing the high-frequency power supplied to the side of the electrodes during a period when the blood flow velocity or blood flow volume is small.
